(19) United States
(12) Patent Application Publication
(10) Pub. No.: US 2015/0147414 A1
(43) Pub. Date: May 28, 2015

(54) FRUCTUS LIGUSTRI LUCIDI FOR GROWTH IN CHILDREN

(71) Applicant: NESTEC S.A., Vevey (CH)

(72) Inventors: Liya Yan Denney, Cambridge (GB); Haotian Feng, Beijing (CN); Pengling Zhao, Beijing (CN); Yangfeng Hou, Beijing (CN); Yujie Shi, Beijing (CN)

(21) Appl. No.: 14/402,545

(22) PCT Filed: May 21, 2013
(86) PCT No.: PCT/EP2013/060409

§ 371 Date: Nov. 20, 2014

(30) Foreign Application Priority Data
May 22, 2012 (CN) PCT/CN2012/075871

(51) Int. Cl. A61K 36/638; A23K 1/16; A23L 1/30

(52) U.S. Cl. CPC A61K 36/638; A23L 1/3002; A23K 1/16; A23V 2002/00
USPC 424/725

(57) ABSTRACT

The present invention relates to a non-therapeutic and therapeutic use of Fructus Ligustri Lucidi for increasing the body growth and in particular body length of young subjects. A further aspect of the invention pertains to food products comprising an alcohol extract of said Fructus as well as pet food products comprising said Fructus.

The present invention relates to a non-therapeutic and therapeutic use of Fructus Ligustri Lucidi for increasing the body growth and in particular body length of young subjects. A further aspect of the invention pertains to food products comprising an alcohol extract of said Fructus as well as pet food products comprising said Fructus.

Body height of a person is determined by a complex interaction of many factors including genetic factors, nutrition and overall health. Physical growth and growth rates also depend on age and vary over a lifetime. In the first year of life, an infant usually grows in length (and also in weight) at an astonishingly rate. But already at age 1, a child's growth in length slows considerably, and by year 2 growth in height usually continues at a fairly steady rate per year until adolescence. However, no child grows at a perfectly steady rate throughout this period of childhood. There are always periods with slightly slower growth alternating with periods with some accelerated growth in length. In average, girls reach their physical adult body height by the age of about 15 years, while boys reach this stage by about 16-17 years of age.

Many people, and particularly young people, wish that they were larger and dream that they were as tall as e.g. their favourite professional sports idol or fashion model. Many times also parents are much concerned about the ‘small’ body height of their child and wish that he or she would grow faster, be if for the child for example to be less teased by other children about its small body size, be it for supporting the child's self-esteem, or even to may be provide the child with a better chance or opportunity for starting his or her future adult professional and personal life. It is a well recognised fact that the physical property of a person including the body height is in today's modern society a factor which has a certain, non neglect-able influence in the professional as well as personal success of a person.

A major determinant factor of height is how long the longest bones become during growth, such as for example the femur, tibia or fibula. Long bones have growth plates, areas that provide space for elongation of the bones. When growth is complete, usually between 15 to 17 years of age in a human, the growth plates close and the bones cannot get any longer.

A number of hormones are involved with the normal development and hence ultimate height of a person. These hormones include growth hormone, thyroid hormone, cortisol and the sex hormones estrogen and testosterone. Nutrition is a further important factor which can drastically influence growth and ultimate height of a person. For example it is well known that malnourished children experience a delayed growth development and often are of a short stature. A well-balanced and appropriate diet maintained throughout childhood is recommended together with appropriate physical activity for a good development of a person's physical height and stature. Furthermore, it has been reported that supplementation of a diet with specific functional ingredients may be helpful and support growth of length of a person.

For example, WO03/037103 discloses a specific food for puppies comprising methionine, sulphur amino acids and dietary lipids for maximising body weight and length gains of growing up puppies.

CN101780265 discloses a health-care food comprising whey protein peptides that can promote increased growth of body length.

WO11/028062 discloses a composition for increasing height, the composition comprising horse bone extract as an active ingredient.

There is still a persisting need with consumers to find alternative and/or better solutions which can be used in or as food products to support and increase the growth in height of young subjects, particularly of children and young adolescents.

The object of the present invention is to improve the state of the art and to provide a new solution for increasing the body growth, in particular body length, of young subjects.

The object of the present invention is achieved by the subject matter of the independent claims. The dependent claims further develop the idea of the present invention.

Accordingly, the present invention provides in a first aspect a non-therapeutic use of Fructus Ligustri Lucidi for increasing the body growth of a young subject.

A second aspect of the invention pertains to a food product or a food supplement comprising an alcohol extract from Fructus Ligustri Lucidi.

A further aspect of the invention pertains to a pet food product comprising Fructus Ligustri Lucidi.

A still further aspect of the invention pertains to the Fructus Ligustri Lucidi for use in the treatment of the medical condition ‘Failure to thrive’(FTT).

It has been surprisingly found by the inventors that when an extract of Fructus Ligustri Lucidi is provided in the food to young weaning female rats during their first 4 months of life, i) the total body weight of those animals was increased, ii) the dry weight, length and diameter of the femurs of those animals were increased, iii) the level of the insulin growth-like factor-1 (IGF-1) in the serum of those animals was significantly increased, and iv) the total body length of those animals was increased, in comparison to a control group kept under same conditions and same food supply but without the above mentioned extract. In particular, the weaning rats used in the experiment were randomized into groups already four weeks after birth and then fed with the supplemented food for the experimental period which lasted 12 weeks. These 1 to 4 months of age of those animals tested correspond to about 3 to 12 years of human age (Australian Rat Information Forum). The rats of the experimental group received a diet supplemented with about 700 mg/kg body weight of a Fructus Ligustri Lucidi ethanol extract per day. Body weight, body length and food intake of the rats were weekly monitored. After 12 weeks of the study period the rats were sacrificed, the femurs collected and analysed. Details of the animal study and obtained results are further disclosed under the Example section below.

The present invention pertains to a non-therapeutic use of Fructus Ligustri Lucidi for increasing the body growth of a young subject, particularly for increasing the growth of the body length of said subject.

“Fructus Ligustri Lucidi” also known as glossy pivet fruit is the dried ripe fruit of Ligustrum lucidum (Oleaceae family), a wild-growing ever-green small tree native in China and Korea. The dried ripe fruit is the part of the plant used in traditional Chinese Medicine.

“Body length” refers to the body height of a person or the total body length of an animal.

Preferably, the non-therapeutic use of the invention pertains to the body growth of a young subject, wherein the increased growth of the body or body length is linked to an increased level of IGF-1 (insulin-like growth factor-1) in the blood serum of the subject.

“IGF-1 (Insulin-like growth factor-1)”, also called somatomedin C, is a hormone similar in molecular structure to insulin. It plays an important role in childhood growth: a synthetic analogue of IGF-1, mecasermin is used for the treatment of growth failure. IGF-1 is a primary mediator of the effects of growth hormone. It stimulates systemic body growth and has a growth-promoting effect on most cells in the body, including skeletal bone and muscle cells. Deficiency of either growth hormone or IGF-1 therefore results in diminished stature.

The present invention further relates to the growth in length of bones which is increased.

The present invention further relates to a young subject, wherein the young subject is a child or growing-up adult from 1 to 20 years of age, preferably from 3 to 18 years of age or preferably from 3 to 15 years of age. Particularly, the young subject is still in the growing-up phase and at an age where he or she has not yet reached the ultimate adult height of his body.

In a further embodiment, the invention relates to a young animal, preferably a young cat or a young dog. Preferably, the young subject is in the growth phase.

In a preferred embodiment of the invention, the Fructus Ligustri Lucidi is provided in the form of an extract, preferably an extract with an alcohol, even more preferably an extract with ethanol. The dried fruit of Fructus Ligustri Lucidi is not very attractive in a direct use in a food product for human consumption because of organoleptic reasons. However, the active ingredients can be extracted from the Fructus Ligustri Lucidi dried fruits by extraction for example with water or alcohol. Thereby, a water extract of those fruits in the form of a medical tea or decoction is well known mainly from the traditional Chinese medicine. The extraction of the active ingredients from those fruits with an alcohol, and particularly with ethanol, seems to be more efficient than the extraction with water and allows to more concentrating those still undefined active ingredients for consumption. The extraction and concentration with ethanol has thereby the advantage that it provides a very efficient extraction method for those active ingredients and that the use of ethanol for extraction purposes for use in the manufacturing of food products is very safe and well accepted by today's consumers.

In a further preferred embodiment of the invention, the Fructus Ligustri Lucidi is provided in a dose of from 4 to 20 g/day, preferably from 6 to 15 g/day to a young subject. The identified dose of from 4 to 20 g/day or preferably from 6 to 15 g/day of the original fruit provides sufficient active ingredient from the Fructus Ligustri Lucidi to a young subject for the claimed health benefit to be effective. Preferably, the Fructus Ligustri Lucidi is provided in the form of an extract, wherein the extract is adjusted to a dose which is equivalent to a consumption of 4 to 20 g/day, preferably of 6 to 15 g/day of said Fructus fruit.

In a further preferred embodiment of the invention, the Fructus Ligustri Lucidi is provided in combination with calcium. As it has been found by the inventors and disclosed herein, that Fructus Ligustri Lucidi improves a.o. the incorporation of calcium into the bones of young subjects. It is therefore advantageous to provide the necessary and sufficient level of calcium to said subjects directly together with the Fructus fruit as part for example of the daily diet. Preferably, the calcium is thereby provided in a daily dose of from 500 to 1′500 mg, preferably from 800 to 1′300 mg.

In a further aspect, the invention pertains to a food product or a food supplement comprising an alcohol extract from Fructus Ligustri Lucidi. This has the advantage that the food product does not need to contain whole fruit matter of the Fructus Ligustri Lucidi per se which is not very attractive to human consumption because of taste and other organoleptic properties of these fruits. Instead, an alcohol extract of said fruit product comprises the active ingredient in a concentrated form which can be easily added or incorporated into a food product or food supplement without affecting its taste or organoleptic quality too much. Preferably, the alcohol is not present anymore in the extract when the extract is used in the manufacturing for example of a food product. This has the advantage that such a food product can be safely used also for younger children below the age of 16 years. Therefore, the invention pertains also to a food product or food supplement comprising an alcohol extract of Fructus Ligustri Lucidi, wherein the alcohol extract is in a dry or powdered form.

A still further aspect of the invention pertains to a pet food product comprising Fructus Ligustri Lucidi. For many animals the taste or organoleptic property of the whole fruit of Fructus Ligustri Lucidi may not be a problem for consumption. Advantageously, pet food products comprising such Fructus whole fruit matters can be produced more easily and cheaper as when first extracts from the Fructus fruit need to be prepared and then incorporated into such pet foods.

In a yet still further aspect, the invention also pertains to the Fructus Ligustri Lucidi for use in the treatment of the medical condition ‘Failure to thrive’ (FTT).

“Failure to thrive (FTT)” is a medical term used in both pediatric and adult human medicine, as well as veterinary medicine. As used by pediatricians, the term covers poor physical growth of any cause and does not imply abnormal intellectual, social or emotional development. Sometimes the term FTT is replaced by the term “faltering growth”.

Those skilled in the art will understand that they can freely combine all features of the present invention disclosed herein. In particular, features described for the non-therapeutic use of the present invention may be combined with the medical use of the present invention and vice versa. Further, features described for different embodiments of the present invention including the food products and supplements may be combined.

Further advantages and features of the present invention are apparent from the example.

### EXAMPLE 1

Weaning female Sprague-Dawley (SD) rats, purchased from the Department of Laboratory Animal Science, Peking University Health Science Center, were used for the experiment. The animals were of specific pathogen-free (SPF) degree. They were housed in temperature and humidity controlled rooms (22±2° C., 50-60% humidity) with a 12 h:12 h light/dark cycle. Animals were acclimatized for one week before formal experiment. After one week, animals were randomized into 2 groups (12 per group):
A) Group 1: fed by AIN-93G diet. This Group is control group.
B) Group 2: fed by AIN-93G diet with Fructus Ligustri Lucidi (FLL) ethanol extract (700 mg/kgBW)

Animals were provided deionised sterile water throughout the research. Both food and water were administered ad libitum. During the experimental period, the body weight, the length of the body and food intake was measured once a week. After 12 weeks, rats were placed into metabolic cages for 4 days: 1-day adaptation and 3-day sample collections of urine and feces. At the end of the metabolic experiment, blood was withdrawn from arteria femoralis under the anaesthesia with Pentobarbital sodium (30 mg/kg BW), and then the rats were sacrificed. Serum was prepared and stored at −80° C. for biochemical determinations. The femur and tibia of both sides were collected, cleaned of all soft tissue, and stored at −20° C. for analysis.

### Methods

### Analysis of Serum Markers of Bone Metabolism:

The serum calcium and phosphorus content was determined by colorimetry using commercially available test kits (Sigma, St Louis, Mo., USA). Insulin-like growth factor-1 (IGF-1) was assessed using the ELISA kit (R&D Ltd, USA) according to the manufacturer's direction.

### Dry Weight, Length and Diameter of Bones:

The femur and tibia from right side were heated in oven at 105° C. to constant weight and the dry weight was measured by analytical balance. The length and diameter at mid-point location of femurs were measured by a vernier caliper.

### Results

### Body Weight Gain

The results indicate that as of week 10 there was a clear trend of body weight gain of the rats fed with FLL (Table 1).

TABLE 1
Average body weight of the rats in g

| Group | 1 w | 2 w | 3 w | 4 w | 5 w | 6 w |
|---|---|---|---|---|---|---|
| Control | 44.74 ± 7.71 | 76.24 ± 15.14 | 111.65 ± 19.22 | 153.32 ± 17.02 | 186.74 ± 18.00 | 212.86 ± 18.85 |
| FLL | 44.69 ± 7.08 | 75.54 ± 16.33 | 108.22 ± 25.18 | 153.88 ± 26.52 | 187.66 ± 24.86 | 212.21 ± 24.55 |

| Group | 7 w | 8 w | 9 w | 10 w | 11 w | 12 w |
|---|---|---|---|---|---|---|
| Control | 231.76 ± 21.94 | 248.76 ± 21.47 | 258.58 ± 24.72 | 267.83 ± 25.43 | 269.65 ± 27.15 | 276.92 ± 35.56 |
| FLL | 231.91 ± 31.96 | 251.54 ± 29.47 | 255.85 ± 36.53 | 268.72 ± 33.69 | 281.01 ± 33.94 | 293.48 ± 31.69 |

### Increase in Serum Level IGF-1

Serum IGF-1 of the FLL group was significantly higher than that of control group (Table 2).

TABLE 2
Concentration of serum IGF-1

| Group | IGF-1 (ng/mL) |
|---|---|
| Control | 530.37 ± 205.44 |
| FLL | 703.21 ± 206.47* |

Note:
Values are expressed as mean ± SD.
*P < 0.05 versus control

### Dry Weight, Length and Diameter of Bones

The femoral dry weight of FLL group was significantly higher than that of the control group (Table 3).

TABLE 3
Effect of FLL extracts on the dry weight and size of femurs

| | Dry weight(g) | length(cm) | diameter (cm) |
|---|---|---|---|
| Control | 0.59135 ± 0.04885 | 3.445 ± 0.206 | 0.440 ± 0.034 |
| FLL | 0.66069 ± 0.04633** | 3.575 ± 0.084 | 0.449 ± 0.021 |

Values are expressed as mean ± SD.
**P < 0.01 versus control

### Body Length

There was a tendency of body length increase similar as to the observed body weight gain. After the 12 weeks period rats fed with FLL had a slightly longer body length in comparison to the control group (Table 4).

TABLE 4
Effect of FLL on body length of rats on week twelve

| | Body length (cm) |
|---|---|
| | Mean value |
| Control | 21.88 |
| FLL | 21.97 |

1. A method for increasing the body growth of a young subject comprising the steps of administering a non-therapeutic amount of Fructus Ligustri Lucidi.

2. Method of claim 1, for increasing the growth of the body length of the subject.

3. Method of claim 1, wherein the increased growth of the body or body length is linked to an increased level of insulin-like growth factor IGF-1 in the blood serum of the subject.

4. Method of claim 1, wherein the length growth of bones is increased.

5. Method of claim 1, wherein the young subject is a child or growing-up adult from 1 to 20 years of age.

6. Method of claim 1, wherein the young subject is a young animal.

7. Method of claim 1, wherein the young subject is in the growth phase.

8. Method of claim 1, wherein the Fructus Ligustri Lucidi is provided in the form of an extract.

9. Method of claim 1, wherein the alcohol is ethanol.

10. Method of claim 1, wherein the Fructus Ligustri Lucidi is provided in a dose of from 4 to 20 g/day.

11. Method of claim 1, wherein the Fructus Ligustri Lucidi is provided in combination with calcium.

12. Method of claim 1, wherein the calcium is provided in a daily dose of from 500 to 1,500 mg.

13. A food product or foods supplement comprising an alcohol extract from Fructus Ligustri Lucidi.

14. A pet food product comprising Fructus Ligustri Lucidi.

15. A method for the treatment of the medical condition ‘Failure to thrive’ comprising administering Fructus Ligustri Lucidi to an individual having same.
